(19) United States
(12) Patent Application Publication
(10) Pub. No.: US 2023/0086671 A1
(43) Pub. Date: Mar. 23, 2023

(54) DEPLOYABLE RADIO-FREQUENCY ABLATION NEEDLE

(71) Applicant: Medtronic Holding Company Sàrl, Tolochenaz (CH)

(72) Inventor: Calin Druma, Sunnyvale, CA (US)

(73) Assignee: Medtronic Holding Company Sàrl

(21) Appl. No.: 17/477,926

(22) Filed: Sep. 17, 2021

(51) Int. Cl. A61B 18/14

(52) U.S. Cl. CPC A61B 18/1477; A61B 2018/00577

(57) ABSTRACT

A deployable radio-frequency (RF) ablation needle is provided. The deployable RF ablation needle is used to apply RF energy to hard and/or soft tissues to facilitate ablation thereof. Portions of the deployable RF ablation needle are configured for expansion from an undeployed configuration to a partially or completely deployed configuration via actuation by a user. The undeployed configuration of these portions of the deployable RF ablation needle affords a relatively small insertion size to facilitate insertion thereof into the hard and/or soft tissues, and the expansion of these portions from the undeployed configuration to the partially or completely deployed configuration correspondingly increases the application area of the RF energy to correspondingly increase the ablation zone afforded by use thereof.

### FIELD

The present technology generally relates to a deployable radio-frequency (RF) ablation needle or probe affording a relatively small insertion size to facilitate insertion thereof into hard and/or soft tissues, and, after insertion, affording reconfiguration from an undeployed configuration to a deployed configuration that expands an end portion thereof to correspondingly increase the size of an ablation zone.

### BACKGROUND

Nerve pain and spinal metastases can be common causes of severe pain among patients with back pain. RF ablation can be used to treat these common causes of severe back pain. Standard RF ablation techniques (without cooling) and cooled RF ablation techniques are options to facilitate such ablation. Needles/probes used for standard RF ablation are generally smaller than needles/probes used for cooled RF ablation. However, ablation zones created using standard RF ablation techniques typically are smaller than ablation zones created using cooled RF ablation techniques. As such, standard RF ablation techniques can require precise placement of the electrodes thereof to be effective. Previously, larger needles/probes and longer ablation times have been employed to increase the size of ablation zones using standard RF ablation techniques and correspondingly mitigate the need for such precise placement. Nevertheless, there remains a need to minimize the size of the needles/probes used for standard RF ablation techniques, while simultaneously affording the creation of larger ablation zones. As discussed below, a deployable RF ablation needle according to the present disclosure is reconfigurable between an undeployed configuration that affords a relatively small insertion size to facilitate insertion into hard and/or soft tissues, and a deployed configuration that expands an end portion of the deployable RF needle to correspondingly increase the size of the ablation zone.

### SUMMARY

The techniques of this disclosure generally relate to a deployable RF ablation needle.

In one aspect, the present disclosure provides a deployable radio-frequency (RF) ablation needle for penetrating into hard and/or soft tissues of a patient, and ablating portions of the hard and/or soft tissues, the deployable RF ablation needle including a proximal end, an opposite distal end, a length between the proximal end and the distal end, and a mid-longitudinal axis extending through the proximal end and the distal end, and along the length of the deployable RF ablation needle; an outer portion extending from at least adjacent the proximal end toward the distal end, the outer portion including an interior cavity defined by an interior first surface; an intermediate portion extending from at least adjacent the proximal end toward the distal end, the intermediate including an exterior second surface and an interior cavity defined by an interior third surface, the intermediate portion terminating at an expandable end portion adjacent the distal end, the expandable end portion including at least two tines moveable between an undeployed configuration and a partially or completely deployed configuration; and an inner portion including a body portion and a head portion, the body portion extending from at least adjacent the proximal end to the head portion, and the head portion extending from the body portion to the distal end, the body portion including an exterior fourth surface; where portions of the intermediate portion are received within the interior cavity of the outer portion, and portions of the body portion of the inner portion are received within the interior cavity of the intermediate portion; where each of the at least two tines includes a first inner surface portion formed as a portion of the interior third surface, and the head portion includes a rearward-facing inclined surface contactable to the first inner surface portions of the at least two tines; and where the head portion is moveable between a first position and a second position, with the second position being closer to the proximal end than the first position, and movement of the head portion from the first position toward the second position contacts the rearward-facing inclined surface with the first inner surface portions, and interaction of the rearward-facing inclined surface and the first inner surface portions expands the expandable end portion by forcing the at least two tines apart from another from the undeployed configuration toward the partially or completely deployed configuration.

In another aspect, the present disclosure provides a deployable radio-frequency (RF) ablation needle for penetrating into hard and/or soft tissues of a patient, and ablating portions of the hard and/or soft tissues, the deployable RF ablation needle including a proximal end, an opposite distal end, a length between the proximal end and the distal end, and a mid-longitudinal axis extending through the proximal end and the distal end, and along the length of the deployable RF ablation needle; an outer portion extending from at least adjacent the proximal end toward the distal end; an intermediate portion extending from at least adjacent the proximal end toward the distal end, a portion of the intermediate portion being received in the outer portion, the intermediate portion terminating at an expandable end portion adjacent the distal end, the expandable end portion including at least four tines moveable between an undeployed configuration and a partially or completely deployed configuration, each of the at least four tines including a first inner surface; and an inner portion including a body portion and a head portion, the body portion extending from at least adjacent the proximal end to the head portion, and the head portion extending from the body portion to the distal end, a portion of the body portion of the inner portion being received in the intermediate portion, and the head portion including a rearward-facing inclined surface; where the head portion is moveable between a first position and a second position, with the second position being closer to the proximal end than the first position, and movement of the head portion from the first position toward the second position contacts the rearward-facing inclined surface with the first inner surface portions, and interaction of the rearward-facing inclined surface and the first inner surface portions expands the expandable end portion by forcing the at least four tines apart from another from the undeployed configuration toward the partially or completely deployed configuration; and where, when the at least four tines are in the undeployed configuration, the distal ends of a first one and a second one of at least four tines are opposite from and spaced apart from one another a first distance in a direction perpendicular to the mid-longitudinal axis, and, when the at least four tines are in the partially or completely deployed configuration, the distal ends of the first one and the second one of the at least four tines are opposite from and spaced apart from one another a second distance in the direction perpendicular to the mid-longitudinal axis, the second distance being greater than the first distance.

In yet another aspect, the present disclosure provides a deployable radio-frequency (RF) ablation needle for penetrating into hard and/or soft tissues of a patient, and ablating portions of the hard and/or soft tissues, the deployable RF ablation needle including a proximal end, an opposite distal end, a length between the proximal end and the distal end, and a mid-longitudinal axis extending through the proximal end and the distal end, and along the length of the deployable RF ablation needle; an outer portion extending from at least adjacent the proximal end toward the distal end; an intermediate portion extending from at least adjacent the proximal end toward the distal end, a portion of the intermediate portion being received in the outer portion, the intermediate portion terminating at an expandable end portion adjacent the distal end, the expandable end portion including at least four tines moveable between an undeployed configuration and a partially or completely deployed configuration, each of the at least four tines including a first inner surface portion; and an inner portion including a body portion and a head portion, the body portion extending from at least adjacent the proximal end to the head portion, and the head portion extending from the body portion to the distal end, a portion of the body portion of the inner portion being received in the intermediate portion, and the head portion including a rearward-facing inclined surface; where the head portion is moveable between a first position and a second position with the second position being closer to the proximal end than the first position, and movement of the head portion from the first position toward the second position contacts the rearward-facing inclined surface with the first inner surface portions, and interaction of the rearward-facing inclined surface and the first inner surface portions expands the at least four tines of the expandable end portion; and where expansion of the at least four tines of the expandable end portion correspondingly increases an ablation zone in the hard and/or soft tissues surrounding the at least four tines with the ablation zone having a first size when the expandable end portion is in the undeployed configuration, and the ablation zone having a second size when the expandable end portion is in the partially or completely deployed configuration, the second size being larger than the first size.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a front, side perspective view of a deployable RF ablation needle according to an embodiment of the present disclosure with an end portion thereof in an undeployed configuration;

FIG. 2 is a side elevational view of the deployable RF ablation needle of FIG. 1 with the end portion thereof in the undeployed configuration;

FIG. 3 is a side cross-sectional view of the deployable RF ablation needle of FIG. 1 with the end portion thereof in the undeployed configuration taken along Line 3-3 of FIG. 2;

FIG. 4 is an enlarged front elevational view of the deployed ablation needle of FIG. 1 with the end portion thereof in the undeployed configuration;

FIG. 5 is an enlarged cross-sectional view of the deployed ablation needle of FIG. 1 depicting a portion of FIG. 3;

FIG. 6 is a front, side perspective view of the deployable RF ablation needle of FIG. 1 in a deployed configuration;

FIG. 7 is a side elevational view of the deployable RF ablation needle of FIG. 1 with the end portion thereof in the deployed configuration;

FIG. 8 is a side cross-sectional view of the deployable RF ablation needle of FIG. 1 with the end portion thereof in the deployed configuration taken along Line 8-8 of FIG. 2;

FIG. 9 is an enlarged front elevational view of the deployed ablation needle of FIG. 1 with the end portion thereof in the deployed configuration; and

FIG. 10 is an enlarged cross-sectional view of the deployed ablation needle of FIG. 1 depicting a portion of FIG. 8.

### DETAILED DESCRIPTION

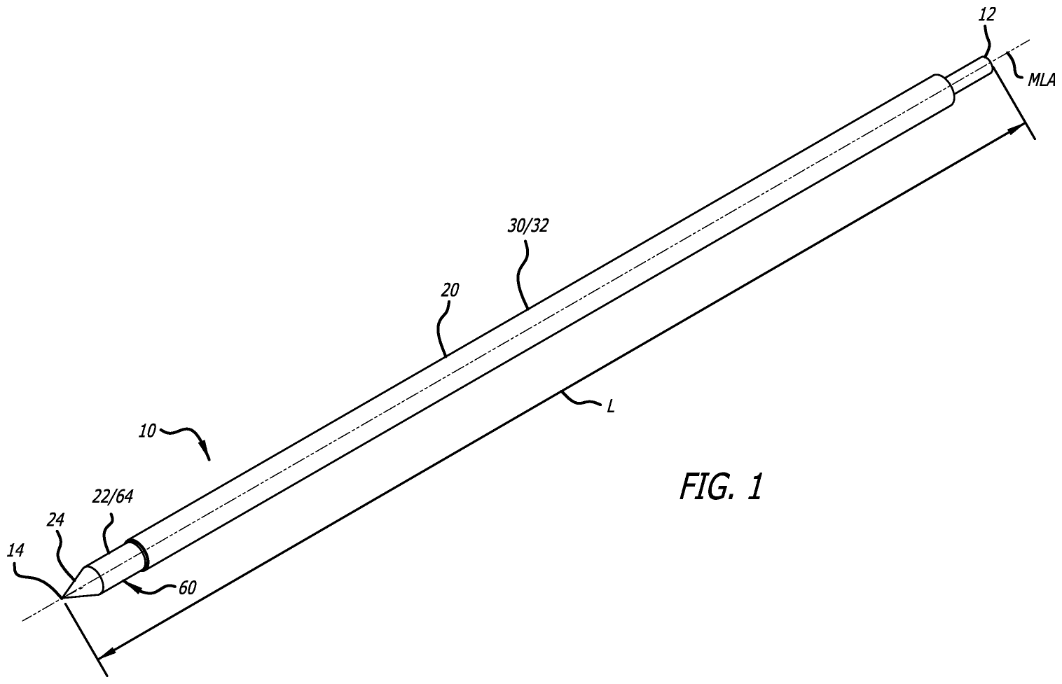

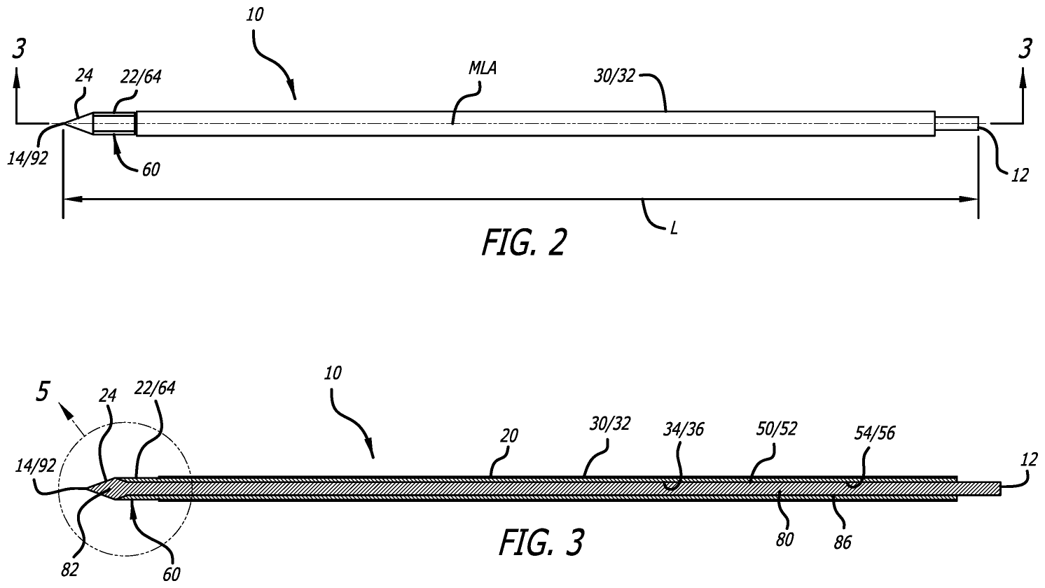

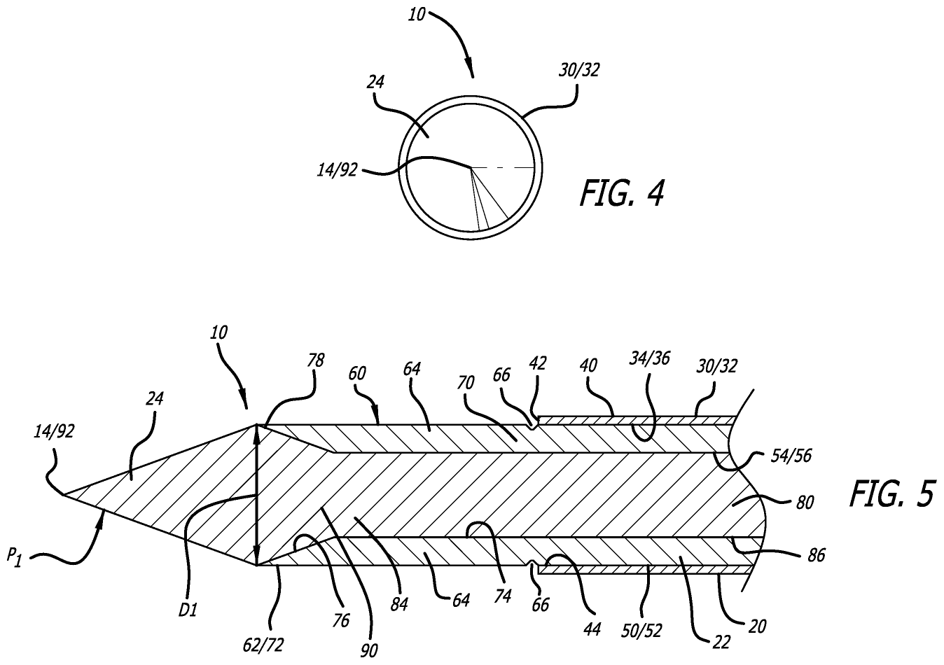

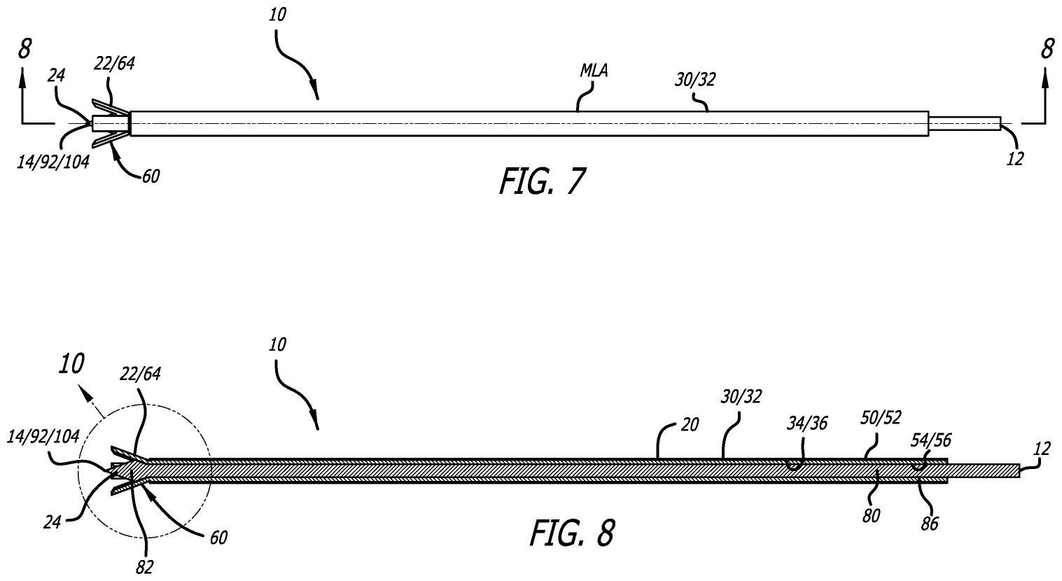

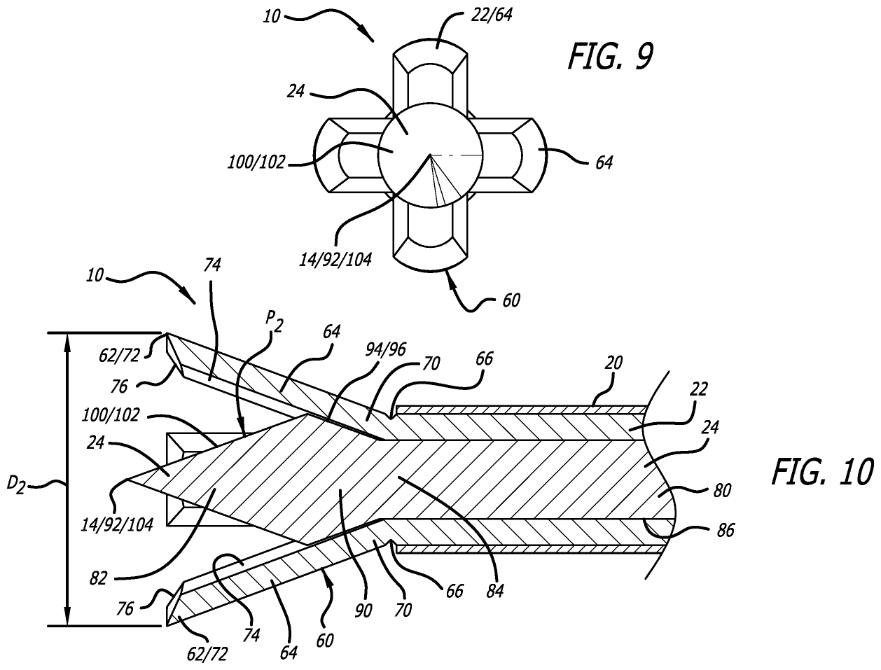

A deployable RF ablation needle in accordance with a preferred embodiment of the present disclosure is generally indicated by the numeral 10 in FIGS. 1-10. As discussed below, the deployable RF ablation needle 10 is used to apply radio-frequency (RF) energy to hard and/or soft tissues to facilitate ablation thereof. Portions of the deployable RF ablation needle 10 are configured for expansion from an undeployed configuration to a partially or completely deployed configuration via actuation by a user. The deployable RF ablation needle 10 can be a monopolar or a bipolar ablation probe, and, as depicted in FIGS. 1-10 is a monopolar ablation probe.

The undeployed configuration of these portions of the deployable RF ablation needle 10 affords a relatively small insertion size to facilitate insertion thereof into the hard and/or soft tissues, and the expansion of these portions from the undeployed configuration to the partially or completely deployed configuration correspondingly increases the application area of the RF energy to correspondingly increase the ablation zone afforded by use thereof. Once these portions of the deployable RF ablation needle 10 are positioned adjacent the hard and/or soft issues requiring ablation, these portions can be expanded from the undeployed configuration to the partially or completely deployed configuration via actuation by the user, and thereafter, the user can activate ablation of the hard and/or soft tissues via application of the RF energy thereto through these portions.

As depicted in FIGS. 1-3 and 6-8, the deployable RF ablation needle 10 includes a proximal end 12, a distal end 14, a mid-longitudinal axis MLA extending through the proximal end 12 and the distal end 14, and a length L along the mid-longitudinal axis MLA. A handle portion (not shown) can be provided at and/or adjacent the proximal end 12 to afford manipulation and/or activation of the deployable RF ablation needle 10 by the user. Operation of the deployable RF ablation needle 10 can also be controlled at the proximal end 12 with or without using the handle portion. The deployable RF ablation needle 10, as depicted in FIGS. 3, 5, 8, and 10, includes an outer portion 20, an intermediate portion 22, and an inner portion 24. The outer portion 20 and the intermediate portion 22 each can extend from at least adjacent the proximal end 12 toward the distal end 14, and the inner portion 24 can extend from at least adjacent the proximal end 12 to the distal end 14.

As discussed below, the outer portion 20 can be configured as a collar, and can surround portions of the intermediate portion 22 and the inner portion 24; the intermediate portion 22 can include or be formed as one or more tines, and the intermediate portion 22 can surround portions of the inner portion 24; and the inner portion 24 can be configured as a trocar. As such, portions of the inner portion 24 extend within portions of the intermediate portion 22, and portions of the intermediate portion 22 extend within portions of the outer portion 20. As discussed below, portions of the inner portion 24 (and/or the outer portion 20) can be moved with respect to the intermediate portion 22.

As depicted in FIGS. 1-3 and 6-8, the outer portion 20 includes a tubular body portion 30 having an exterior first surface 32, an interior surface 34, and a first passageway 36 defined by the interior second surface 34 and formed through the body portion 30. The body portion 30 can be made of an insulative material including, for example, polyimide(PI), PET, PETG, PEEK, or other thermoplastics, as well as PTFE. The body portion 30 terminates at an end portion 40 thereof having a distal end 42, and a first aperture 44 into the first passageway 36 is formed in the end portion 40 at the distal end 32. Some portions of the intermediate portion 22, as depicted in FIGS. 3, 5, 8, and 10, are received in the first passageway 36, and some portions of the intermediate portion 22 extend outwardly from the first aperture 44 toward the distal end 14. As discussed below, at least some portions of the outer portion 20 can be moveable relative to the intermediate portion 22 to facilitate exposure of portions of the intermediate portion 22.

As depicted in FIGS. 3 and 5, the intermediate portion 22 includes a body portion 50 having an exterior third surface 52, an interior fourth surface 54, and a second passageway 56 defined by the interior fourth surface 54 and formed through the body portion 50. The exterior third surface 52 is sized and shaped to fit through the first aperture 44 and within the first passageway 36, and the exterior third surface 52 can interface with the interior second surface 34. The body portion 50 can be made of a conductive material including, for example, stainless steel, titanium, or other biocompatible metals. The body portion 50 terminates at an expandable end portion 60 thereof having a distal end 62. The second passageway 56 extends through the expandable end portion 60, when the expandable end portion 60 is unexpanded. As discussed below, all or portions of the expandable end portion 60 can serve as the electrode of the deployable RF ablation needle.

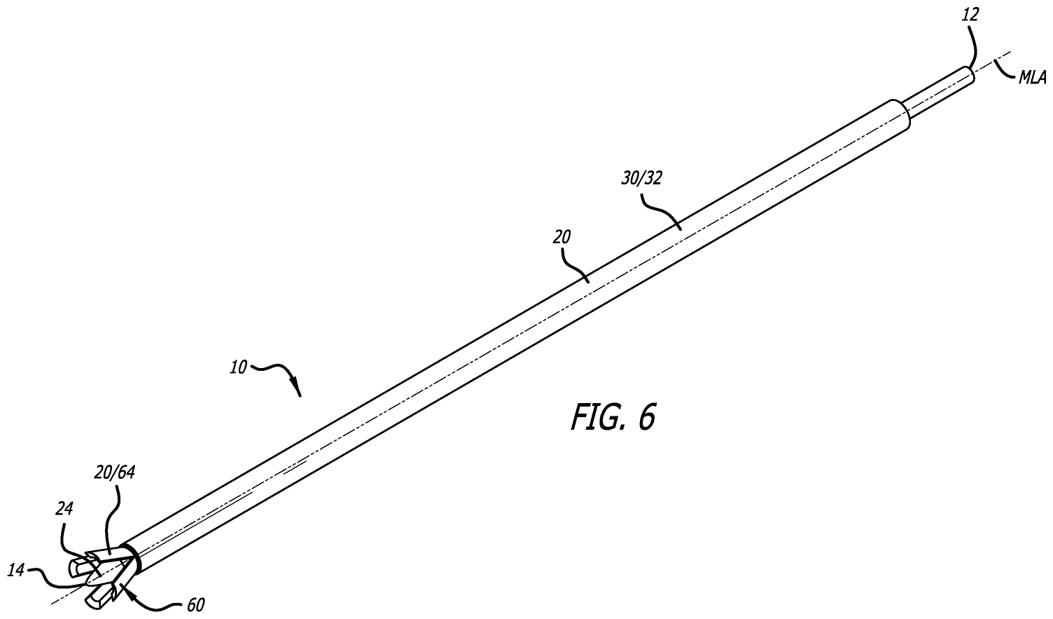

The expandable end portion 60 can be formed from multiple tines 64, and all or portions the tines 64 can each serve as an electrode of the deployable RF ablation needle 10. As depicted in FIGS. 6, 7, and 9, the expandable end portion 60 can be formed by four (4) equally sized tines 64 that can divide the end portion 60 into quarters. However, the expandable end portion 60 can be formed, for example, by two (2) tines 64 dividing the expandable end portion 60 into two parts, by three (3) tines 64 dividing the expandable end portion 60 into three parts, by five (5) tines 64 dividing the expandable end portion 60 into five parts, and by six (6) tines 64 dividing the expandable end portion 60 into six parts. The tines 64, whether two, three, four, five, six, etc., can be formed of equally or differently sized portions of the expandable end portion 60.

The tines 64 can be moveable between an undeployed configuration (FIGS. 1-5) to a deployed configuration (FIGS. 6-10) to expand and open the expandable end portion 60. The tines 64 can be unbiased or biased toward the undeployed configuration, and the tines 64 can be forced apart from one another to move the tines 64 from undeployed configuration toward the deployed configuration to expand the expandable end portion 60 relative to the mid-longitudinal axis MLA. To facilitate such expansion, portions of the body portion 50 are scored in areas 66 on the exterior third surface 52 to facilitate outward bending of the tines 64 relative to the mid-longitudinal axis MLA. As such, each of the tines 64 can be attached to remaining portions of the intermediate portion 22 at or adjacent the scorings 66.

Each of the tines 64 can include a proximal end 70, a distal end 72, a first surface portion 74 (that is part of the interior fourth surface 54), and a second surface portion 76 (that is also part of the interior fourth surface 54), with the proximal ends 70 being located adjacent a corresponding one of the areas 66, and the distal ends 72 being collocated with the distal end 62. The scorings at the areas 66 can be formed by indentations formed in the exterior third surface 52 of the body portion 50 that form pivot points affording each of the tines 64 to bend outwardly when pressure is applied thereto. In the undeployed configuration (FIGS. 1-5), the distal ends 72 have a minimum undeployed dimension D₁ (FIG. 5) between one another in a direction perpendicular to the mid-longitudinal axis, and in the deployed configuration (FIGS. 6-10), the distal ends 72 have a maximum deployed dimension D₂ (FIG. 10) between one another in a direction perpendicular to the mid-longitudinal axis.

A second aperture 78 (FIG. 5) into the second passageway 56 is formed at the distal end 62 of the expandable end portion 60 when the expandable end portion 60 is unexpanded. Some portions of the inner portion 24, as depicted in FIGS. 1-5, can be received in the second passageway 56, and some portions of the inner portion 24 extend outwardly from the second aperture 78 to the distal end 14. As discussed below, at least some portions of the inner portion 22 can be moveable relative to the intermediate portion 22 to apply force to the tines 64, and the force applied by these portions of the inner portion 24 against the first surface portions 74 and the second surface portions serves to move the tines 64 from the undeployed configuration toward the deployed configuration by bending the tines 64 outwardly at the scorings at the areas 66. As such, the aperture 78 formed at the distal end 62 ceases to exist due to movement of the tines 64 from the undeployed configuration toward the deployed configuration. Furthermore, the scorings at the areas 66 can also serve as catches that prevent movement of the outer portion 20 and the intermediate portion 22 relative to one another.

As depicted in FIGS. 3, 5, 8, and 10, the inner portion 24 includes a body portion 80 and a head portion 82. The body portion 80 and the head portion 82 can be made of an insulative material including, for example, PI, PET, PETG, PEEK, or other thermoplastics, as well as PTFE. The body portion 80 includes a distal end 84 and an exterior fifth surface 86. The exterior fifth surface 86 is sized and shaped to fit through the second aperture 78 and within the second passageway 56, and the exterior fifth surface 86 can interface with the interior fourth surface 54. The head portion 82 includes a proximal end 90 and a distal end 92, and can be attached at the proximal end 86 to the distal end 84 of the body portion 80. Furthermore, the head portion 82 includes a first portion 94 with a rearward-facing inclined surface or surfaces 96 and a second portion 100 with a forward facing surface or surfaces 102. As discussed below, the head portion 82 is moveable between a first position P₁ (FIGS. 1-5) and a second position P₂ (FIGS. 6-10) relative to the intermediate portion 22, the rearward-facing inclined surface(s) 96 of the first portion 94 forms a wedge that is used in forcing the tines 64 apart from one another to expand and open the expandable end portion 60, and the forward-facing surface(s) 102 second portion 100 serve as cutting edge(s) to facilitate insertion of the deployable RF ablation needle 10 into the hard and/or soft tissues.

The rearward-facing inclined surface(s) 96 can have frusto-conical shape(s) for contacting the tines 64. Furthermore, the forward-facing surface(s) 102, as depicted in FIG. 10, terminate at a tip portion 104, and the forward-facing surface 102 can have conical or frusto-conical shape(s). The tip portion 104 and the shape(s) of the forward-facing surface(s) 102 afford penetration of the deployable RF ablation needle 10 through the hard and/or soft tissue. Such penetration affords positioning of the head portion 82 and the tines 64 adjacent an area of the hard and/or soft tissues requiring ablation. As depicted in FIG. 5, the first surface portion 74 of the tines 64 can be shaped to conform to the shape(s) of the body portion 80, and/or the second surface portions 76 of the tines 64 can be shaped to conform to the shape(s) of the rearward-facing inclined surface(s) 96, such that, when the head portion 82 is in the first position P₁, the minimum undeployed dimension D₁ between the distal ends 72 correspond to the maximum dimension of the head portion 82 in a direction perpendicular to the mid-longitudinal axis MLA. As such, at least portions the tines 64 adjacent the distal ends 72 are tucked behind portions of the head portion 82 to aid ease of penetration of the deployable RF ablation needle 10 into the hard and/or soft tissues.

As depicted in FIG. 10, the rearward-facing inclined surface(s) 96 contact the second surface portions 76 then the first surface portions 74 of the tines 64 as the head portion 82 is moved from the first position P₁ toward the second position P₂. Given the shape(s) of the rearward-facing inclined surface(s) 96, such contact forces the tines 64 further and further apart from one another (from the minimum undeployed dimension D₁ toward the maximum deployed dimension D₂) during movement of the head portion 82 from the first position P₁ (FIG. 5) toward the second position P₂ (FIG. 10). As such, when the head portion 82 is moved toward the proximal ends 70 of the tines 64 (and toward the distal end 42 of the end portion 40 of the body portion 30), the tines 64 are forced apart from one another to expand the distal ends 72 thereof from the minimum undeployed dimension D₁ toward the maximum deployed dimension D₂. Ultimately, movement of the head portion 82 is stopped by interactions of portions the intermediate portion 22 with portions of the end portion 40 of the body portion 30 that block such movement. The tines 64 are positioned at the maximum deployed dimensions D₂ and the expandable end portion 60 can be fully expanded when such movement is stopped.

As discussed above, the tines 64 serve as electrodes, and the size of the ablation zone of the deployable RF ablation needle 10 is dependent on the amount of expansion of the tines 64 of the expandable end portion 60. The expansion of the expandable end portion 60 relative to the mid-longitudinal axis MPA increases the application area of the RF energy to correspondingly increase the ablation zone afforded by use of the deployable RF ablation needle 10. In other words, the larger the expansion of the tines 64 due to movement of the head portion 82 from the first position P₁ toward the second position P₂, the larger the ablation zone. As such, the user can control the size of the ablation zone by selecting the amount of expansion of the tines 64 of the expandable end portion 60.

Once the tines 64 are expanded to a selected amount, electrical current can be applied to the ablation zone via the tines 64. The application of the electrical current, for example, can be controlled by the user with or without use of the handle. To illustrate, as discussed above, the intermediate portion 22 (including the tins 64) can be formed from a conductive material. The intermediate portion 22 (including the tines 64) can be electrically connected to an electrical-current generator (not shown) using one or more wires (not shown), and operation of the electrical-current generator can be controlled by the user at the handle. The amount of electrical current applied through the tines 64 to the hard and/or soft tissues can be adjusted via adjustment by the user of the electrical-current generator, for example, at the generator itself and/or at the handle. Thus, via adjustment and activation/deactivation thereof, the user can control amount(s) and/or duration(s) of the electrical current applied to the hard and/or soft tissues via the tines 64. Thus, the size of the ablation zone can be controlled via the amount of expansion of the tines 64 of the expandable end portion 60, and the amount(s) and/or the duration(s) of the electrical current applied to the hard and/or soft tissues through the tines 64. The ablation zone can be sized to encompass all or portions of the hard and/or soft tissues requiring ablation.

While the shapes of the body portion 30, the body portion 50, and the body portion 80 are depicted in FIGS. 1-10 as being substantially cylindrical, the shapes thereof are not so limited. The body portion 30, the body portion 50, and the body portion 80 can have any number of cross-sectional shapes such as, for example, rectangular, triangular, oval shapes provided that portions of the body portion 80 can fit inside the body portion 50, and portions of the body portion 50 can fit inside the body portion 30.

In addition to the inner portion 22 being moveable relative to the intermediate portion 22, the outer portion 20 can be moveable relative to the intermediate portion 22 to facilitate covering, then uncovering and exposing the tines 64. To illustrate, the body portion 30 of the outer portion 20 can be moveable to position the distal end 42 of the end portion 40 at or adjacent the distal ends 72 of the tines 64. In doing so, the distal end portion 40 can cover all or portions of the tines 64 to aid ease of penetration of the deployable RF ablation needle 10 into the hard and/or soft tissues. Thereafter, the distal end portion 40 can be moved to uncover and expose the tines 64.

In addition to bending outwardly due to force applied by the head portion 82 against the tines 64, the tines 64, rather than being unbiased or biased toward the undeployed configuration, can be biased to toward the deployed configuration to bend outwardly relative to the mid-longitudinal axis MLA. Thus, after movement of the outer portion 20 away from the distal ends 72 that uncovers the tines 64 (after being formerly covered by the distal end portion 40), the tines 64 can bend outwardly due to such bias. The head portion 82 of the inner portion 24 can then be moved, as described above, from the first position P₁ toward the second position P₂ to further expand the expandable end portion 60.

During use thereof, the deployable RF ablation needle 10 is inserted by the user into a patient to ultimately position the expandable end portion 60 (and the tines 64) adjacent the hard and/or soft tissues requiring ablation. The user can manipulate the placement of the end portion 60 with or without use of the handle. Once the tines 64 are positioned adjacent the hard and/or soft tissues requiring ablation, the user can manipulate the deployable RF ablation needle 10 move the head portion 82 of the inner portion 24 from the first position P₁ toward the second position P₂. In doing so, the tines 64 are expanded to expand the distal ends 72 thereof from the minimum undeployed dimension D₁ toward the maximum deployed dimension D₂. If the outer portion 20 is moveable relative to the intermediate portion 22 to facilitate covering then exposure of the tines 64, the outer portion 20 first would need to be moved to uncover and expose all or portions of the tines 64 to allow expansion thereof.

The user can select the amount of expansion of the tines 64 to control the size of the ablation zone via the amount of movement of the head portion 82 from the first position P₁ toward the second position P₂. Furthermore, the user can also control the size of the ablation zone 60 by adjusting the amount(s) and/or activating/deactivating the duration(s) of the electrical current applied to the hard and/or soft tissues through the tines 64. After ablation of the hard and/or soft tissues, the insertion process can be repeated in reverse to facilitate removal of the deployable RF ablation needle 10 from the patient.

As discussed above, the undeployed configuration of the tines 64 affords a relatively small insertion size to facilitate insertion thereof into the hard and/or soft tissues, and the expansion of the tines 64 from the undeployed configuration to a partially or completely deployed configuration correspondingly increases the application area of the RF energy to correspondingly increase the ablation zone afforded by use thereof.

It should be understood that various aspects disclosed herein may be combined in different combinations than the combinations specifically presented in the description and the accompanying drawings. It should also be understood that, depending on the example, certain acts or events of any of the processes of methods described herein may be performed in a different sequence, may be added, merged, or left out altogether (e.g., all described acts or events may not be necessary to carry out the techniques). In addition, while certain aspect of this disclosure are described as being performed by a single module or unit for purposes of clarity, it should be understood that the techniques of this disclosure may be performed by a combination of units or modules associated with, for example, a medical device.

I/We claim:

1. A deployable radio-frequency (RF) ablation needle for penetrating into hard and/or soft tissues of a patient, and ablating portions of the hard and/or soft tissues, the deployable RF ablation needle comprising:
a proximal end, an opposite distal end, a length between the proximal end and the distal end, and a mid-longitudinal axis extending through the proximal end and the distal end, and along the length of the deployable RF ablation needle;
an outer portion extending from at least adjacent the proximal end toward the distal end, the outer portion including an interior cavity defined by an interior first surface;
an intermediate portion extending from at least adjacent the proximal end toward the distal end, the intermediate including an exterior second surface and an interior cavity defined by an interior third surface, the intermediate portion terminating at an expandable end portion adjacent the distal end, the expandable end portion including at least two tines moveable between an undeployed configuration and a partially or completely deployed configuration; and
an inner portion including a body portion and a head portion, the body portion extending from at least adjacent the proximal end to the head portion, and the head portion extending from the body portion to the distal end, the body portion including an exterior fourth surface;
wherein portions of the intermediate portion are received within the interior cavity of the outer portion, and portions of the body portion of the inner portion are received within the interior cavity of the intermediate portion;
wherein each of the at least two tines includes a first inner surface portion formed as a portion of the interior third surface, and the head portion includes a rearward-facing inclined surface contactable to the first inner surface portions of the at least two tines; and
wherein the head portion is moveable between a first position and a second position, with the second position being closer to the proximal end than the first position, and movement of the head portion from the first position toward the second position contacts the rearward-facing inclined surface with the first inner surface portions, and interaction of the rearward-facing inclined surface and the first inner surface portions expands the expandable end portion by forcing the at least two tines apart from another from the undeployed configuration toward the partially or completely deployed configuration.

2. The deployable RF ablation needle of claim 1, wherein at least portions of each of the at least two tines serves as an electrode for applying electrical current to the hard and/or soft tissues to facilitate ablation thereof.

3. The deployable RF ablation needle of claim 1, wherein the rearward-facing inclined surface is at least in part frusto-conical, and the rearward-facing inclined surface serves as a wedge to force the at least two tines apart from one another.

4. The deployable RF ablation needle of claim 1, wherein, when the at least two tines are in the undeployed configuration, the distal ends of a first one and a second one of at least two tines are and spaced apart from one another a first distance in a direction perpendicular to the mid-longitudinal axis, and, when the at least two tines are in the partially or completely deployed configuration, the distal ends of the first one and the second one of the at least two tines are spaced apart from one another a second distance in the direction perpendicular to the mid-longitudinal axis, the second distance being greater than the first distance.

5. The deployable RF ablation needle of claim 4, wherein expansion of the expandable end portion correspondingly increases an ablation zone in the hard and/or soft tissues surrounding the at least two tines with the ablation zone having a first size when the first one and the second one of the at least two tines are spaced apart the first distance, and the ablation zone having a second size when the first one and the second one of the at least two tines are spaced apart the second distance, the second size being larger than the first size.

6. The deployable RF ablation needle of claim 5, wherein each of the at least two tines includes a proximal first end and a distal second end, the proximal first ends being pivotally attached to remaining portions of the intermediate portion to facilitate movement of the at least two tines between the undeployed configuration and the partially or completely deployed configuration.

7. The deployable RF ablation needle of claim 5, wherein at least portions of each of the at least two tines serves as an electrode for applying electrical current to the hard and/or soft tissues to facilitate ablation thereof.

8. The deployable RF ablation needle of claim 1, wherein the head portion includes a forward-facing surface configured to facilitate penetration of the deployable RF ablation needle into the hard and/or soft tissues, and the forward-facing surface terminates at a tip collocated with the distal end of the deployable RF ablation needle.

9. The deployable RF ablation needle of claim 8, wherein, when the at least two tines are in the undeployed configuration, portions of the at least two tines are tucked behind portions of the head portion in the direction of insertion.

10. The deployable RF ablation needle of claim 9, wherein each of the at least two tines includes a second inner surface angled to correspond to the rearward-facing inclined surface to facilitate contact therebetween when the at least two tines are in the undeployed configuration.

11. A deployable radio-frequency (RF) ablation needle for penetrating into hard and/or soft tissues of a patient, and ablating portions of the hard and/or soft tissues, the deployable RF ablation needle comprising:
a proximal end, an opposite distal end, a length between the proximal end and the distal end, and a mid-longitudinal axis extending through the proximal end and the distal end, and along the length of the deployable RF ablation needle;
an outer portion extending from at least adjacent the proximal end toward the distal end;
an intermediate portion extending from at least adjacent the proximal end toward the distal end, a portion of the intermediate portion being received in the outer portion, the intermediate portion terminating at an expandable end portion adjacent the distal end, the expandable end portion including at least four tines moveable between an undeployed configuration and a partially or completely deployed configuration, each of the at least four tines including a first inner surface; and
an inner portion including a body portion and a head portion, the body portion extending from at least adjacent the proximal end to the head portion, and the head portion extending from the body portion to the distal end, a portion of the body portion of the inner portion being received in the intermediate portion, and the head portion including a rearward-facing inclined surface;
wherein the head portion is moveable between a first position and a second position, with the second position being closer to the proximal end than the first position, and movement of the head portion from the first position toward the second position contacts the rearward-facing inclined surface with the first inner surface portions, and interaction of the rearward-facing inclined surface and the first inner surface portions expands the expandable end portion by forcing the at least four tines apart from another from the undeployed configuration toward the partially or completely deployed configuration; and
wherein, when the at least four tines are in the undeployed configuration, the distal ends of a first one and a second one of at least four tines are opposite from and spaced apart from one another a first distance in a direction perpendicular to the mid-longitudinal axis, and, when the at least four tines are in the partially or completely deployed configuration, the distal ends of the first one and the second one of the at least four tines are opposite from and spaced apart from one another a second distance in the direction perpendicular to the mid-longitudinal axis, the second distance being greater than the first distance.

12. The deployable RF ablation needle of claim 11, wherein at least portions of each of the at least four tines serves as an electrode for applying electrical current to the hard and/or soft tissues to facilitate ablation thereof.

13. The deployable RF ablation needle of claim 11, wherein the rearward-facing inclined surface is at least in part frusto-conical, and the rearward-facing inclined surface serves as a wedge to force the at least four tines apart from one another.

14. The deployable RF ablation needle of claim 11, wherein expansion of the expandable end portion correspondingly increases an ablation zone in the hard and/or soft tissues surrounding the at least four tines with the ablation zone having a first size when the first one and the second one of the at least four tines are spaced apart the first distance, and the ablation zone having a second size when the first one and the second one of the at least four tines are spaced apart the second distance, the second size being larger than the first size.

15. The deployable RF ablation needle of claim 14, wherein each of the at least four tines includes a proximal first end and a distal second end, the proximal first ends being pivotally attached to remaining portions of the intermediate portion to facilitate movement of the at least four tines between the undeployed configuration and the partially or completely deployed configuration.

16. The deployable RF ablation needle of claim 14, wherein at least portions of each of the at least four tines serves as an electrode for applying electrical current to the hard and/or soft tissues to facilitate ablation thereof.

17. A deployable radio-frequency (RF) ablation needle for penetrating into hard and/or soft tissues of a patient, and ablating portions of the hard and/or soft tissues, the deployable RF ablation needle comprising:
a proximal end, an opposite distal end, a length between the proximal end and the distal end, and a mid-longitudinal axis extending through the proximal end and the distal end, and along the length of the deployable RF ablation needle;
an outer portion extending from at least adjacent the proximal end toward the distal end;
an intermediate portion extending from at least adjacent the proximal end toward the distal end, a portion of the intermediate portion being received in the outer portion, the intermediate portion terminating at an expandable end portion adjacent the distal end, the expandable end portion including at least four tines moveable between an undeployed configuration and a partially or completely deployed configuration, each of the at least four tines including a first inner surface portion; and
an inner portion including a body portion and a head portion, the body portion extending from at least adjacent the proximal end to the head portion, and the head portion extending from the body portion to the distal end, a portion of the body portion of the inner portion being received in the intermediate portion, and the head portion including a rearward-facing inclined surface;
wherein the head portion is moveable between a first position and a second position with the second position being closer to the proximal end than the first position, and movement of the head portion from the first position toward the second position contacts the rearward-facing inclined surface with the first inner surface portions, and interaction of the rearward-facing inclined surface and the first inner surface portions expands the at least four tines of the expandable end portion; and
wherein expansion of the at least four tines of the expandable end portion correspondingly increases an ablation zone in the hard and/or soft tissues surrounding the at least four tines with the ablation zone having a first size when the expandable end portion is in the undeployed configuration, and the ablation zone having a second size when the expandable end portion is in the partially or completely deployed configuration, the second size being larger than the first size.

18. The deployable RF ablation needle of claim 17, wherein at least portions of each of the at least four tines serves as an electrode for applying electrical current to the hard and/or soft tissues to facilitate ablation thereof.

19. The deployable RF ablation needle of claim 17, wherein the rearward-facing inclined surface is at least in part frusto-conical, and the rearward-facing inclined surface serves as a wedge to force the at least four tines apart from one another.

20. The deployable RF ablation needle of claim 17, wherein each of the at least four tines includes a proximal first end and a distal second end, the proximal first ends being pivotally attached to remaining portions of the intermediate portion to facilitate movement of the at least four tines between the undeployed configuration and the partially or completely deployed configuration.
